(19) United States
(12) Patent Application Publication
(10) Pub. No.: US 2018/0272050 A1
(43) Pub. Date: Sep. 27, 2018

(54) MEDICAL DEVICES HAVING SURFACE MODIFIERS

(71) Applicant: ANGIODYNAMICS, INC., Latham, NY (US)

(72) Inventors: Raymond LAUREAU, Westford, MA (US); Benjamin Bell BELL, Shrewsbury, MA (US)

(21) Appl. No.: 15/991,961

(22) Filed: May 29, 2018

Related U.S. Application Data
(63) Continuation of application No. 14/886,969, filed on Oct. 19, 2015.
Application No. 14/886,969 is a continuation of application No. 14/216,036, filed on Mar. 17, 2014, now abandoned.
(60) Provisional application No. 61/786,849, filed on Mar. 15, 2013.
(60) Provisional application No. 61/787,905, filed on Mar. 15, 2013.

(51) Int. Cl. A61M 1/00; C08L 83/04; A61M 1/36; C08L 27/12; A61L 29/06; A61L 29/08; B29C 47/00; A61M 25/00; A61M 39/06; B29L 31/00; A61M 39/02; B29K 27/12; B29K 83/00

(52) U.S. Cl. CPC A61M 1/008; A61M 2039/0273; A61M 1/3659; C08L 27/12; A61M 1/3673; A61L 29/06; A61L 29/085; B29C 47/0066; A61M 2025/0046; B29C 47/0023; A61M 2210/12; A61M 2207/00; A61M 2205/0238; A61M 2039/064; B29L 2031/7548; A61M 2039/0258; B29C 2793/0036; B29K 2027/12; B29K 2083/00; B29L 2031/7542; C08L 83/04

(57) ABSTRACT

Improved medical devices having anti-thrombogenic and anti-adherent surface modifiers for improved medical device performance and patient outcomes are provided. In certain embodiments, the medical devices are at least partially manufactured using an admixture of a base polymer and surface modifying fluoropolymer additives. In certain embodiments, the medical devices are vascular access devices, vascular access accessories, peripheral vascular devices, or components of these devices.

### CROSS-REFERENCE TO RELATED APPLICATIONS

This application is a continuation of U.S. application Ser. No. 14/218,036, filed Mar. 17, 2014, entitled “Medical Devices Having Surface Modifiers,” which claims priority to U.S. Provisional Patent Application Ser. No. 61/787,905 filed Mar. 15, 2013, entitled “Improved Medical Devices Having Surface Modifiers,” and U.S. Provisional Patent Application Ser. No. 61/786,849 filed Mar. 15, 2013, entitled “Variable Characteristic Venous Access Catheter Shaft with Surface Modifiers and Methods,” each of which is incorporated herein by reference for all purposes.

### FIELD OF THE INVENTION

The present invention relates generally to medical devices having anti-thrombogenic and anti-adherent surface modifiers for improved medical device performance.

### BACKGROUND OF THE INVENTION

Medical device related thrombus poses a serious health risk for patients, resulting in a clinical challenge for medical professionals. Thrombus occurs on a medical device when blood components (including platelets and thrombus) adhere and accumulate on the device. For indwelling devices, challenges include a potential for device related complications, compromised blood flow dynamics, inability to aspirate or infuse through the device, or potential for thrombo-emboli. If the surface of a medical device does not allow blood components to adhere, then thrombus accumulation can be minimized. When thrombus accumulation is minimized, device performance and patient safety can be improved.

Catheters are a common type of vascular access device used to gain fluid access to a target site within the body. While a distal portion of the catheter is indwelling, it is often desirable to cut-off access to an external outdwelling portion of the catheter by sealing the catheter lumen closed. If a catheter lumen is left open, the patient risks infection as well as embolisms due to air entering the blood stream. Clamps, clips and other types of compression elements are commonly used on catheter extension tubing to seal the catheter lumen closed. However, compression elements can potentially damage the device as forces required to seal the catheter tubing lumen are dispersed over a small surface area and can damage the tubing wall.

As an alternative to external compression devices, pressure activated safety valves have been used to seal vascular access catheters when the catheter is not in use, such as the BioFlo PICC with PASV valve technology (AngioDynamles, Inc., Latham, N.Y.). Safety valves with anti-adherent coatings have been described in U.S. Pat. No. 8,187,234 to Weaver et al, incorporated herein by reference. However, manufacturing obstacles and performance issues arise when using an anti-adherent coating on a flexible valve element. First, it is difficult to manufacture a flexible valve element so that it has a homogenously distributed coating on all surfaces of the flexible membrane. For instance, from a manufacturing perspective, it would be-very difficult to evenly and fully coat the opposing interior surfaces of the slit. Further, even if a specialty process existed, such a process could be expensive and cost prohibitive. The process might also compromise the integrity of the flexible valve element if it required propping the slit open during the coating process. Further, the flexible member experiences significant stress during infusion and aspiration procedures since it is designed to flex. High flow rate procedures such as power injection only increase the levels of stress on the flexible valve member. Since the flexible valve element spans across the lumen of the valve housing, it is subjected to the direct force of fluid infusion. These dynamics make a coating prone to flaking, cracking or eroding. Further, the face of the silt and the opposing interior surfaces of the slit are moving parts, and coatings are not very durable on flexing membranes. Thrombus buildup on the valve may lead to compromised device performance and disruption in fluid dynamics within the valve housing. Between infusions, thrombus buildup could also cause the slit to malfunction by either adhering to the outside of the slit and blocking the slit from opening, or becoming lodged between interior surfaces of the slit, propping the slit open.

Medical suites commonly use single lumen or dual lumen PICCs for accessing the vascular system. PICCs are typically selected by French size, or outer diameter. The selection of the type of PICC will depend on the type of treatment being provided, and the physical characteristics of the patient. It is often desirable to use the smallest viable French size, which is commonly a 4 French single lumen PICC or a 5 French dual lumen PICC. These are typically viewed as the smallest viable French size since downsizing any lower will decrease the inner diameter of the lumen to the point that the incidence of thrombus related catheter occlusion will dramatically increase. Thus, for example, the lumen of a conventional 3 French single lumen PICC has a greater potential for thrombus related catheter occlusion in comparison to a 4 French single lumen PICC.

Introducers are a commonly used tool by medical professionals for gaining access to a vessel. Introducers typically consist of two parts; (1) a dilator having a tapered tip for graduated access into the vessel through the site of a venipuncture, and (2) a sheath to accommodate the insertion of a medical device, for example, a dialysis catheter. The dilator and sheath are normally provided as an assembly with the sheath preloaded over the dilator, with at least a portion of the dilator's tapered tip exposed through a distal opening in the sheath. During a procedure, a guidewsre is inserted into the body through a needle, and after the needle is retracted off of the guidewsre, the dilator sheath assembly is loaded over the guldewire and advanced into the target vessel. At this point, the dilator and the guidewire can be retracted, exposing the sheath lumen and providing access for a medical device to be advanced through the lumen and into the target vessel. Trauma caused to the vessel wall often leads to a localized areas of thrombus adhesion and buildup on the sheath at the site of the vessel wall puncture. An introducer with inner sheath layer comprised of a fluoropolymer has been described in publication, including U.S. Pat. No. 5,380,304 to Parker. This layered approach provides the anti-thrombotic properties of fluorine within the introducer lumen, while maintaining kink resistance of the sheath by using a more rigid polymer in the outer layer.

Implantable ports are devices commonly used to access a target site in the vascular system. They are implanted subcutaneously and attached to a port catheter having a tip terminating at the target site tor treatment. The target site is typically within the vascular system, such as the junction of the superior vena cava and the right atrium. The port, has a reservoir sealed by an elastomeric septum, and the reservoir can be accessed by puncturing the septum with a needle. Since the reservoir is in fluid communication with the tip of the port catheter, fluid can be infused to the target site by infusing fluid into the reservoir. Fluid can also be aspirated from the target site by using the needle to create a negative pressure within the reservoir.

A point of concern to medical professionals using implantable ports in their patients is the incidence of thrombus buildup and occlusions. One point of thrombus buildup and occlusion is commonly the port reservoir, a buildup sometimes referred to as sludge. Sludge buildup in the reservoir can lead to increased infection rates and diminished device performance, particularly when attempting to withdraw blood through the port reservoir. Another point of thrombus buildup and occlusion is the tip of the port catheter. A fibrin sheath can form at the tip of the port catheter, narrowing or completely occluding the port catheter lumen, adversely impacting device performance. These issues also impact patient safety since ports often serve as the conduit for critical treatments. Thrumbus may also form at the site of the venipuncture. Further, occlusions lead to increased infection rates, and port occlusions may require additional surgeries if the port needs to he removed or replaced. Healthcare costs also increase when additional procedures have to be performed and additional devices are required.

Systems and methods for removing undesirable material within a circulatory system have been described in publications including U.S. Pat. No. 8,075,510 to Aklog et al., incorporated herein by reference. Such devices are also used by medical professionals, including the AngioVac Cannula (AngioDynamics, Inc., Latham, N.Y.). In an exemplary system, a suction cannula can be used for removing undesirable material from a site of interest in a patient. Undesirable material may include for example a blood clot, emboli, thrombi, DVT, pulmonary embolism, vegetative growth, endocarditis:, cardioembolism or tumors. The material can be removed en bloc facilitated by the creation of a vortex flow through an expanded member at the distal end of the cannula. For larger pieces of undesirable material, it would be advantageous to optimize the rapid movement of the material through the expanded member and through the cannula. In addition, a system as described in U.S. Pat. No. 8,075,510 utilizes a closed extracorporeal circuit for capturing the undesirable material, isolating the undesirable material from the circuit and reinfuslng blood back into the patient. However, the circuit may be prone to partial or full clogging or occlusion since blood traveling outside of the body has a higher tendency to clot as its temperature drops.

Thrombolytic catheters are commonly used in procedures for delivering lytic agents to dissolve blood clots. These types of catheters typically have side holes or slits along their shaft for systematically delivering lytic agents to the clot. An example of a thrombolytic catheter has been described in U.S. Pat. No. 5,250,034 to Appling et al., incorporated herein by reference. For certain thrombolytic catheters utilizing slits, it is optimal for the slits to open uniformly and simultaneously once a threshold pressure is reached within the catheter lumen. However, if platelets and thrombus accumulate on or within the slit, slit function could be affected, compromising the ability for the device to function properly. Further, platelet adhesion within an interior wall of a slit could prop the slit open, interfering with the ability of the device to properly build to a threshold pressure or deliver lytic agents uniformly and simultaneously. Thrombolytic catheters utilizing side holes could also experience deteriorated device performance if one or more side holes become obstructed and lytic agents cannot be uniformly distributed to the target area of the clot.

Drainage catheters, such as biliary and urinary catheters are often used as a conduit for allowing for drainage of a fluid, such as drainage of a patient's urine from the bladder. They typically have one or more side holes at their distal end for providing fluid access to the catheter lumen. However, since these catheters are often inserted and indwelling for long-term use, encrustation can obstruct the side holes, leading to an increased incidence of infection, deterioration of catheter performance and risk to the patient as crystalized formations stuck to the catheter traumatize the patient upon catheter withdrawal.

Many types of medical devices for use within the vasculature system have a lattice, mesh, crosshatch, weave or other similar type of configuration where multiple members are crossing or in tight proximity to one another. Examples of these types of medical devices include stents and filters and venous valves. These types of devices are often designed to be directly within the path of the blood stream, and device performance can be adversely impacted by issues associated with platelet adhesion and thrombus buildup. These issues may also pose significant health risks to the patient, increasing the risk for thrombo-emboli. Further, devices are commonly mounted or centered within the vessel via a sharp point of contact which partially penetrates the vessel wall, mounting the device in place. Thrombus accumulation can buildup on the device at the point of contact since the vessel wall is being traumatized. Several types of non-thrombogenic coatings have been proposed for this class of devices, however, improvements in device performance and improved efficiency in methods of manufacture are still desired.

For the types and classes of medical devices described above, it would be desirable to improve device performance while minimizing health risks to the patient. Additionally, there is a need to produce improved medical devices for fluid communication with the body, where the device can maintain higher performance levels over time. Further, it is desirable to build such devices utilizing a reliable, cost effective and efficient method of manufacture.

### SUMMARY OF THE INVENTION

The invention is directed to medical devices having antl-thrombogenic and anti-adherent surface modifiers. In one aspect, the invention is a medical device at least partially manufactured using an admixture of a base polymer and surface modifying fluoropolymer additives. In certain aspects, the invention is a vascular access device, a vascular access accessory, a peripheral vascular device, or a component of a medical device manufactured using an admixture of a base polymer and surface modifying fluoropolymer additives.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which tike numerals represent like elements, and in which;

FIG. 1A is a top view of a catheter according to a first embodiment of the invention;

FIG. 1B is a perspective partial cross-sectional view of the valve housing of the catheter shown in FIG. 1A with the valve in the open position;

FIG. 1C .is a perspective partial cross-sectional view of the valve housing of the catheter shown in FIG. 1A with the valve in the closed position;

FIG. 2 Is a perspective view of a flexible valve element shown in FIG. 1B and 1C;

FIG. 3 is a cross-sectional view of the flexible valve element shown in FIG. 2;

FIG. 4 is a perspective view of a catheter according to a second embodiment of the invention;

FIG. 5 is a perspective view of a dilator sheath assembly according to a third embodiment of the invention;

FIG. 6 is a perspective view of the dilator shown in FIG. 5;

FIG. 7 is a perspective view of the sheath shown in FIG. 5;

FIG. 8 is a cross-sectional view of a septum according to a fourth embodiment of the invention;

FIG. 9 is a cross-sectional view of an alternative septum according to a fourth embodiment of the invention;

FIG. 10 is a cross-sectional perspective view of a reservoir according to a fourth embodiment of the invention;

FIG. 11 is a cross-sectional perspective view of a floor insert according to a fourth embodiment of the invention;

FIG. 12 is a cross-sectional perspective view of a port assembly according to a fourth embodiment of the invention;

FIG. 13 is a side view of a port catheter according to a fourth embodiment of the invention;

FIG. 14 is a perspective view of a suction cannula according to a fifth embodiment of the invention;

FIG. 15 is a cross-sectional view of a blood circuit according to a fifth embodiment of the invention;

FIG. 16 is a top partial view of a thrombolytic catheter according to a sixth embodiment of the invention;

FIG. 17 is a cross-sectional view of the thrombolytic catheter shown in FIG. 16;

FIG. 18A is a magnified view of a slit from FIG. 16 in closed position;

FIG. 18B is a magnified view of a slit from FIG. 16 in open position;

FIG. 19 is a partial perspective view of the distal end of a catheter according to a seventh embodiment of the invention;

FIG. 20 is a partial cross-sectional view of the catheter shown in FIG. 19;

FIG. 21 is a perspective side view of a cross-pattern for an indwelling medical device according to an eighth embodiment of the invention;

FIG. 22 is a magnified view of the cross-pattern shown in FIG. 21;

FIG. 23 is a plan view of a venous access catheter according to a ninth embodiment of the invention;

FIG. 24 Is a graph showing a % of surface modifier in proximal, transition, and distal segments of the venous access catheter shown in FIG. 23;

FIG. 25 is a graph showing a % of surface modifier and hardness in proximal, transition, and distal segments of the venous access catheter shown in FIG. 23; and

FIG. 26 is a partial perspective view of a dialysis catheter tip.

### DETAILED DESCRIPTION OF THE INVENTION

The present invention can be understood more readily by reference to the following detailed description, the examples included therein, and to the Figures and their following description. The drawings, which are not necessarily to scale, depict selected preferred embodiments and are not intended to limit the scope of the invention. The detailed description illustrates by way of example, not by way of limitation, the principles of the invention. The skilled artisan will readily appreciate that the devices and methods described herein are merely examples and that variations can be made without departing from the spirit and scope of the invention. It is also to be understood that the terminology used herein is for the purpose of describing particular embodiments only and is not intended to be limiting.

Referring now in detail to the drawings, in which like reference numerals indicate like parts or elements throughout the several views, in various embodiments, presented herein are improved medical devices having surface modifiers.

As used herein, “additives” refer to any materials that are added into the polymeric materials of the present invention to influence physical, mechanical, or other material properties, or to advantageously impact manufacturability or desired performance characteristics. Examples of known additives for polymeric materials include pigments (used synonymously herein with colorants), solvents, biostabilizers, plasticizers, nucleating agents fillers, radiopaque powders (or other forms), and materials in any form that enhance biocompatibility or other in vivo performance characteristics. An example of a fluoropolymer additive that is used in embodiments of the present invention is marketed under the trade name ENDEXO (Interface Biologics, Inc., Toronto, Ontario Canada), which generally refers to a fluoropolymer additive material described in U.S. Pat. No. 6,127,507, which is herein incorporated by reference. As used herein, “fluoropolymer” means a fluorocarbon-based polymer, including oligomers, having carbon-fluorine bonds. In a preferred embodiment, the fluoropolymer used in the present invention is a fluoroalkyl fluoropolymer that is characterized by terminal polyfluoro oligomeric groups.

As described in U.S. Pat. No. 6,127,507, this additive may be referred to as a “surface modifying molecule” or “surface modifier.” The surface modifying additives, when used in embodiments of the invention, are preferably synthesized in a manner that they contain a base polymer compatible segment and terminal hydrophobic fluorine components which are non-compatible with the base polymer. The compatible segment of the surface modifier is selected to provide an anchor for the surface modifier within the base polymer substrate upon admixture. While not being bound by theory, it is believed that the fluorine tails are responsible in part for carrying the surface modifier to the surface of the admixture, with the chemical resistant fluorine chains exposed out from the surface. The latter process is believed to be driven by the thermodynamic incompatibility of the fluorine tail with the polymer base substrate, as well as the tendency towards establishing a low surface energy at the mixture's surface. When the balance between anchoring and surface migration is achieved, the surface modifier remains stable at the surface of the polymer, while simultaneously altering surface properties. The utility of the additives of the invention versus other known macromolecular additives, lies in 1) the molecular arrangement of the amphipathic segments in the surface modifier chain, i.e. two-ω fluoro-tails, one at each end, with the polar segment sandwiched between them; 2) the molecular weight of the fluorine tails relative to that of the central segment and; 3) the ability of the materials to inhibit biodegradation of the base polymer when the fluoro-segments are stabilized at the interface, which provides improved blood compatibility and biostability of the base polymers. This latter improvement has not been previously achieved and/or demonstrated with any other family of amphipathic polymeric type surface modifying macromolecules.

The surface modifying macromolecules used in embodiments of the present invention significantly alter the surface chemistry of, for example, segmented polyurethanes, i.e. the surface modifiers present on the surface of the polymer mixture and exhibit a new hydrophobic surface. This new surface carries many of the attributes of perfluoro-carbon chains and, therefore, is significantly more stable with respect to oxidation and hydrolysis than many polyurethanes. Further, the surface has low fouling properties and low wetting characteristics.

The surface modifying additives used in embodiments of the present invention are, for example, of use with linear or crossllnked polyurethane-based materials. By tailoring the central segment components of the surface modifier, the fluoropolymer additives can be applied inter alia to a wide range of polymer materials which include polymers synthesized with reagents that are of common knowledge in the field of polyurethanes.

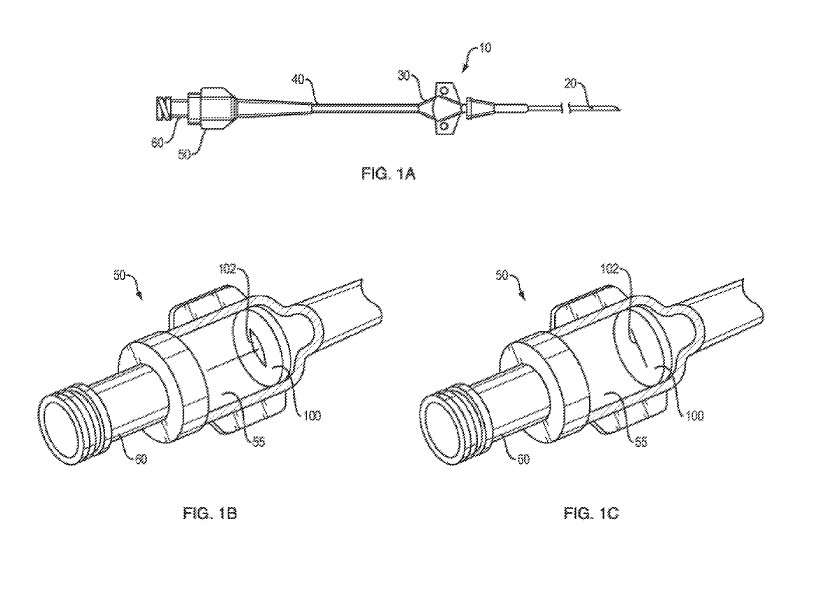

Referring now to FIGS. 1A-1C, a vascular access catheter with a pressure activated safety valve according to a first embodiment of the invention is shown. FIG. 1A shows a single lumen catheter 10 (more specifically, a peripherally inserted central catheter (PICC)) having a catheter shaft 20, a hub 30, an extension tube 40, a valve housing 50 and a luer 60. The catheter shaft 20 in this embodiment is a single lumen catheter shaft, but in alternative embodiments can be a dual, triple or otherwise multi-lumen catheter. Slit valves, a type of pressure activated safety valve includes a disk shaped flexible valve element 100 made of an elastomer such as silicone having one or more slits 102 extending therethrough. The slit can move between an open configuration (as shown in FIG. 1B) and closed configuration (as shown in FIG. 1C) based on a threshold fluid pressure within the lumen 55 of the valve housing 50.

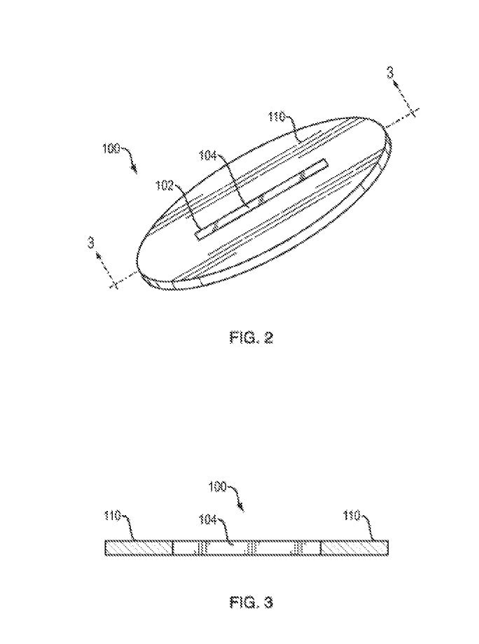

Referring to FIG. 2, the flexible valve element 100 is shown. The flexible valve element 100 is a disk shaped elliptical member having a valve element body 110 and a slit 102 centered along its long axis. The slit 102 is designed to stay closed under normal venous pressure fluctuations, and open In response to a threshold pressure during infusion or aspiration of a fluid. The slit has a pair of interior slit walls (one shown 104 and the other not shown) with dimensions defined by the length of the slit 102 and the thickness of the flexible element 100 at the slit 102. In the eross-sectlonal view shown in FIG. 3, the inferior slit wall 104 is shown with respect to the rest of the valve element body 110. Although the thickness and concavity of the valve element illustrated in FIGS. 2 and 3 are shown to be constant, they can be variable. Further, curved or multiple slit configurations can be implemented as is known in the art.

The flexible valve element 100 of the present invention is manufactured from an admixture of a base polymer and surface modifying fluoropolymer additives. The fluoropolymer additive is integral to the admixture and homogenously distributed throughout the admixture. A base polymer according to a preferred embodiment is silicone. Desired fluorination levels in the flexible valve element 100 may require a 2-25% by weight ratio of additive to base elastomer. Once the desired levels are achieved, the admixture is rolled into sheets using a calendaring process. A disk shaped element can then be cut using a die, and a slit can be punched into the disk to form the desired slit configuration. Alternative methods known in the art for shaping the disk and cutting one or more slits into the disk can also be employed.

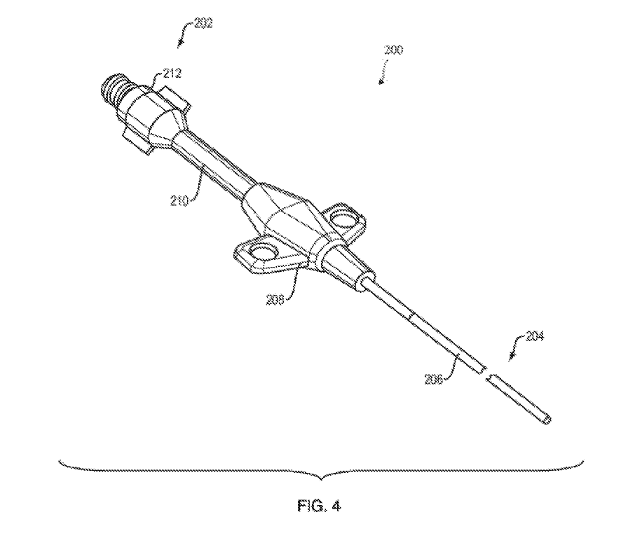

Since the fluoropolymer additive is distributed homogenously throughout the bulk of the disk, the fluoropolymer additive is present on all valve element body 110 surfaces, including the surface of the interior slit wall 104 and the corner where the interior slit wall meets the face of the valve element body 110. The additional and difficult step of applying an antl-thrombogenic coating to the surface of the interior slit wall 104 can be avoided. The flexible valve element of the present embodiment features superior durability since its anli-thrombogenic properties cannot erode or wear over time, as may otherwise be the case with safety valve having an anti-thrombogenic coating. Since the interior walls of the slit are also anti-adherent, the slit can open uniformly after repeated infusion and aspiration procedures, further minimizing wear and tear of the edges of the slit. Additionally, thromboresistant properties can be incorporated into the internal walls of the valve housing securing the flexible valve element. The flexible valve element 100 can be incorporated into a conventional catheter such as that described in FIGS. 1A-1C, or into a catheter having a shaft comprising fluoropolymer additives, such as that described below with respect to FIG. 4 for superior valve performance. In this case, the vaive will be in line with a fluid path including the inner wall of a catheter shaft lumen, the inner wall of the extension tube lumen, the inner wail of the valve housing, and the flexible valve element, all of which can have advantage of durable anti-thombogenic and anti-adherent surfaces.

Referring to FIG. 4, a peripherally inserted central catheter (PICC) 200 is shown. PICC 200 has a proximal 202 and distal 204 end, with a catheter shaft 206 in fluid communication with an extension tube 210 by connection to a hub 208. A luer fitting 212 is connected to the proximal end of the extension tube 210 for connecting to an external device such as a power injector. The catheter shaft 206 is a compound polymer mix of a base polymer and a surface modifying fluoropolymer additive. The additive is present throughout the wail of the catheter shaft 206, and the surface modifier tends to present on exposed surfaces formed during the extrusion process. A similar result happens from manufacture of the extension tube 210. The hub 208 and luer 212 can be formed by an injection molding process, where the compound polymer is injected into a mold to form the component.

Since thrombus will not adhere or buildup on surfaces using these admixtures, catheter infection rates and rates of catheter malfunction due to occlusion can be minimized. For example, using a surface modifier for a 3 French single lumen PICC would allow for a high performance downsized PICC without compromising patient safety associated with thrombus buildup and catheter lumen occlusion. Further, the anti-adherent properties of the inner wall forming the fluid channel reduce the amount of drag on the fluid so that high flow rates can foe maintained. Dual lumen PICC lines could also be further downsized to at least 4 French without compromising performance or patient safety. Single and dual lumen PICC configurations could be even further downsized when able to accommodate the type of treatment being administered. As complication and PICC removal rates decrease, the patient benefits from experiencing less trauma while healthcare costs are minimized.

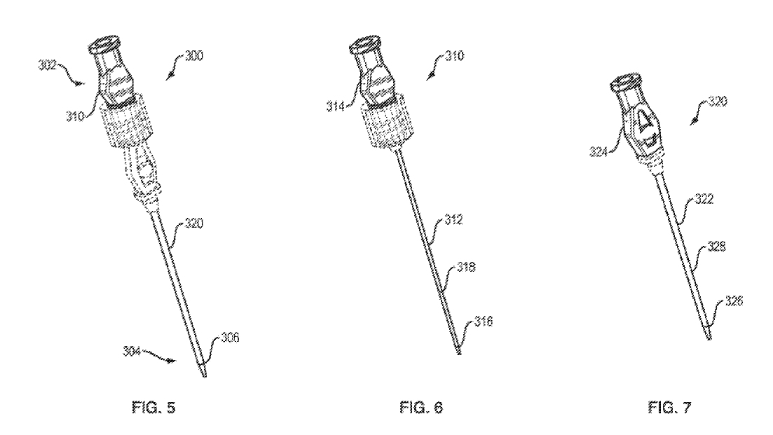

Referring to FIGS. 5-7, a dilator 310, sheath 320 and a dilator sheath assembly 300 forming the introducer system are shown. The dilator sheath assembly has a proximal 302 and distal 304 end. With respect to the dilator shown in FIG. 6, the distal end terminates in a tapered tip 316 for introducing into the site of a venipuncture. The proximal end has a hub 314 which can be used to secure the dilator 310 to the sheath 320 when assembled. A guidewire lumen 318 is formed by the dilator shaft wall 312 and extends through the dilator 310. The Introducer sheath 320 has a proximal end with a hub 324, and distal end terminating in an opening 326 to the lumen 328. When the dilator sheath is assembled, the distal end 318 of the dilator 310 extends slightly from the opening 326 to act as a leading tip, and a transition zone 306 is formed on the assembly where the dilator 310 stops and the introducer sheath 320 begins. A seam can run along the sheath wall to facilitate splitting the sheath once it is ready to be retracted over a medical device such as a dialysis catheter. The introducer sheath wail 322 is a compound polymer mix of a kink-resistant base polymer and a surface modifying fluoropolymer additive.

The additive is integral and homogenously distributed throughout the introducer shaft and the dilator, and the surface modifier tends to present on all surfaces formed during the extrusion pnocess. As a result, the anti-thrombogenic properties are present both on the inner and outer surfaces of the sheath wail 322, preventing thrombus adhesion both within the sheath lumen 328 and on the outer surface of the sheath wall 322 at the site of vessel entry. Further, these anti-thrombotic properties can be achieved in a simple manufacturing process that does not require a more complicated anti-thrombogenic coating approach. Additionally, the anti-adherent inner surfaces of the introducer sheath 320 lumen 328 facilitate less resistance upon advancement upon introduction of a medical device. The anti-adherent outer surfaces of the introducer sheath assembly help the medical professional achieve a smooth transition into the site of the venipuncture and into the vessel with minimal resistance. Specifically, the outer surface of the sheath wall 322, the outer and leading surfaces of the tapered tip 316 on the dilator 310, and all transition zone 308 surfaces between the dilator 310 and the sheath 320 which slide along the puncture site upon insertion will present with the anti-adherence of the surface modifying fluoropolymer additive, leading to a superior and smooth insertion.

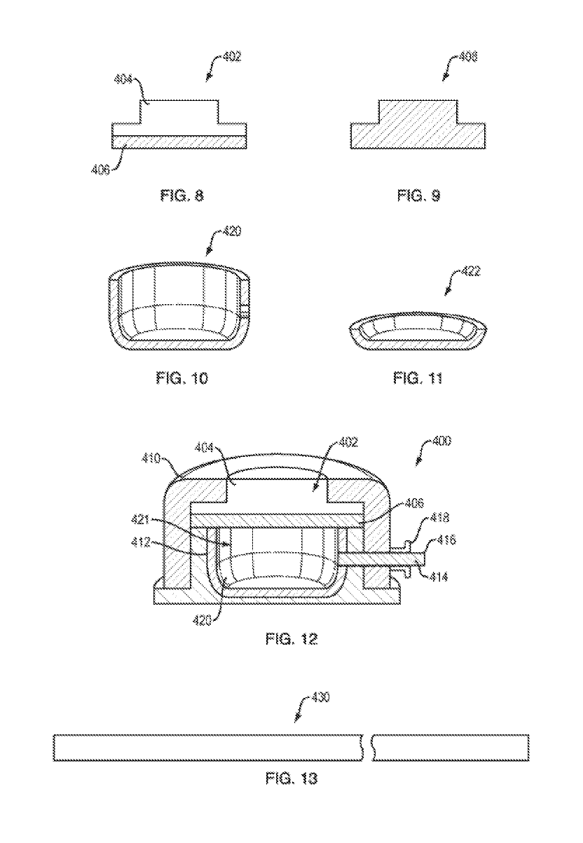

Referring to FIGS. 8-13, components and an assembly for a port catheter system are shown. Illustrated by the port assembly 400 of FIG. 12, the port 400 has a reservoir 421 bounded by a base housing portion 412. The reservoir 421 is fluidly sealed by an elastomeric septum 402 which sits on the base housing portion 412. The septum 402 is secured to the base housing portion 412 by a retaining portion 410. The reservoir 421 is in fluid communication with a stem 418 via an outlet lumen 414. A collar 418 used for securing the port catheter surrounds the stem 416.

The reservoir is lined with an insert 420 composed of an admixture of a rigid base polymer and a surface modifying fluoropolymer additive. To provide an anti-thrombogenic property to the insert 420, fluoropolymer additives are combined in admixture with a base polymer for the manufacture of the insert 420. The fluoropolymer additive is integral to the admixture used in manufacturing the insert 420. As a result, when the insert 420 is manufactured through a polymer forming process known in the art, such as injection molding, the fluoropolymer additive integral and homogenously distributed throughout the admixture is present throughout the insert's bulk and on any surfaces. The anti-thrombotic properties of the insert 420 allow for a decreased incidence of sludge. Alternatively, the entire port housing could be manufactured to include a thromboresistant additive by molding the component from a compound mixture to include the additive. Since the reservoir is resistant to platelet adhesion and thrombus buildup due to the additive, the laminar flow within the port is more consistently maximized, increasing flushing ability and providing a synergistic mechanism for keeping the port reservoir clear of sludge. This property helps to optimize port system performance for high flow procedures such as power injection of contrast fluid. Further, the anti-adherent properties of the surface modifying fluoropolymers help to promote a rapid flushing action, which also helps to prevent sludge.

Another advantage of using an insert is that it has superior durability characteristics compared to an antl-thrombotic coating. Implantable ports are designed for patients with longer term and repeated need for procedures such as power injection of contrast fluid. When infusion needles are inserted into the reservoir, the tip of the needle will commonly contact and scrape the bottom surface. This may happen repeatedly over the lifespan of the port. Power injection of fluid into the reservoir may also tend to facilitate the erosion and wear of a surface coating over time, decreasing the effectiveness of the coating to provide an anti-adherent properly in the reservoir. However, the insert 420 contains the fluoropolymer additive throughout its bulk, and therefore manual abrasion of the insert's surface caused needle scrapes and surface erosion from power injection will not impact anti-thrombotic properties within the reservoir due to the homogenous distribution of the additive throughout the entire thickness of the reservoir insert wall. Alternatively, a floor insert 422 could be customized specifically for covering the bottom surface, which is the area of the reservoir most affected by sludge buildup and scraping from the infusion needle. For ports utilizing a floor insert 422, an anti-thrombotic coating could be used on the side walls of the reservoir. In some embodiments, the floor insert 422 could have a soft durometer so that the tip of an inserted needle can stabilize within the floor insert, providing more control over fluid flow and efficiencies in reservoir fluid dynamics.

As the port fills with fluid or as blood splashes around the port reservoir, it is possible that certain amounts of blood will come into contact with the bottom surface of the septum. The port septum, typically made from a self-sealing elasiomeric polymer such as silicone, may also comprise a fiurorpolymer additive for providing a top surface to the reservoir that is thromboresistant. As illustrated in FIG. 12, this exemplary embodiment insulates the reservoir such that all fluid contacting surfaces of the reservoir are thromboresistant. The lumen of the port stem 414 can also be lined with an anti-thrombotic coating or insert, or be formed of an admixture polymer having surface modifying characteristics. An insert for the port stem 414 can also be integral to a reservoir insert 422, 420 as described above for providing a seamless transition between the reservoir and the stem lumen. As illustrated in FIG. 8 and FIG. 12, the septum can have a lop layer 404 of a base polymer and a bottom layer 406 admixture of a base polymer and a fluoropolymer additive. These layers can be formed separately then fused together, or formed as a two stem injection molding process. Alternatively, as illustrated in FIG. 9, the entire septum can be formed of an admixture of a base polymer and a fluoropolymer additive. However, it may be preferable to use a conventional elastomer in a top layer 404 as shown in FIG. 8 to avoid compromising the self-sealing properties of the septum, further ensuring that the septum retains a high stick count.

Port systems described above can be used in conjunction with a port, catheter 430 shown in FIG. 13. The catheter 430 may be composed of a compound polymer including a fluoropolymer additive. The catheter shaft is a compound polymer mix of a base polymer and a fluoropolymer additive. The additive is present throughout the bulk of the catheter shaft, and the surface modifier tends to present on shear surfaces formed during the extrusion process. Since this admixture is resistant to blood adhesion or thrombus buildup on surfaces of the port catheter, thrombus buildup and occlusion at the tip of the port catheter is minimized and an anti-fouling effect is achieved as device performance is improved, especially during aspiration. Risk to the patient's health and procedure costs are also minimized as the device becomes more reliable. Ease of infusion needle advancement through the septum is also improved since the bulk of the septum features anti-adherent properties, decreasing fractional forces on the shaft of the needle.

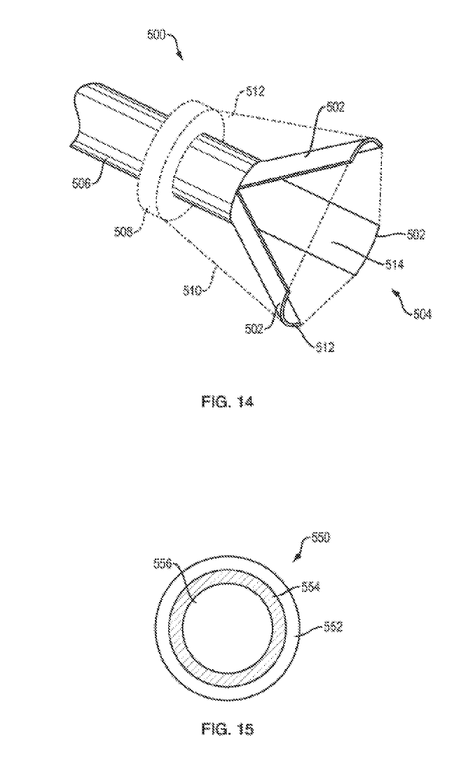

Referring to FIG. 14, the distal end of a suction cannula 500 used in connection with a system for removing undesirable material within a circulatory system is shown. Cannula 506 is cut at its distal end into a number of strips 502 capable of pivoting between an open and closed position. The strips 502 deploy to an open position as the balloon 508 is inflated causing the jacket 510 to become taut or expand. When the strips 502 deploy to an open position, a funnel 504 is formed, which provides expanded space for clot to advance through as suction is applied to the device. The cannula is made from an admixture of a base polymer and a surface modifying fluoropolymer additive.

The fluoropolymer additive is integral to the bulk of the cannula wall, and homogenousily distributed throughout the wall of the cannula shaft. As a result, when the cannula 506 is initially fanned, then subsequently cut at its distal end to form pivoting strips 502, anti-adherent properties are present at the edges 512 and surfaces 514 of the strips 502. These anti-adherent edges 512 and surfaces 514 resist thrombus buildup, further facilitating the rapid en bloc removal of clot, emboli, or thrombi from the target site. Since the fluoropolymer additive is homogenously distributed throughout the cannula wall, strips of various shapes can be easily cut and manufactured without the complicated step of evenly distributing an anti-adherent coating or layer on the edges of the strip. The jacket 510 and balloon 508 can aiso.be composed of an admixture of a flexible polymer (such as flexible polymers known in the art for manufacturing medical balloons) and a fluoropolymer additive to facilitate rapid movement of clot, emboli, or thrombi through the funnel 504. This further adds to the anti-fouling advantage of embodiments of the invention, particularly for sections of the cannula 500 that are reliant on moving parts for an effective procedure. Additionally, these features help to maintain a consistent slow rate throughout the cannula and the system during the procedure. As an added benefit, this anti-adherent feature will facilitate less resistive removal of these components from their molds during manufacture.

Referring to FIG. 15, an external blood circuit 550 is used with the above described thrombus removal system. The circuit 550 has an Inner layer 554 of a compound polymer having a fluoropolymer additive that can be coextruded with a rigid polymer 552 for controlling platelet adhesion and thrombus buildup. The rigid outer layer 552 supports the structure of the circuit keeping the circuit lumen 556 open and the walls 552, 554 from collapsing into the lumen 558 as high negative pressures are exerted within the circuit lumen 556. The anti-adherent properties of the inner layer 554 help to promote and maintain high flow rates and the rapid movement of clot, emboli, or thrombi, through the circuit lumen high. This is beneficial for thrombus removal systems having external circuits since blood may cool while traveling through the external circuit for an extended period of time, thus becoming more prone to clotting. Frictional forces at the wall are also reduced.

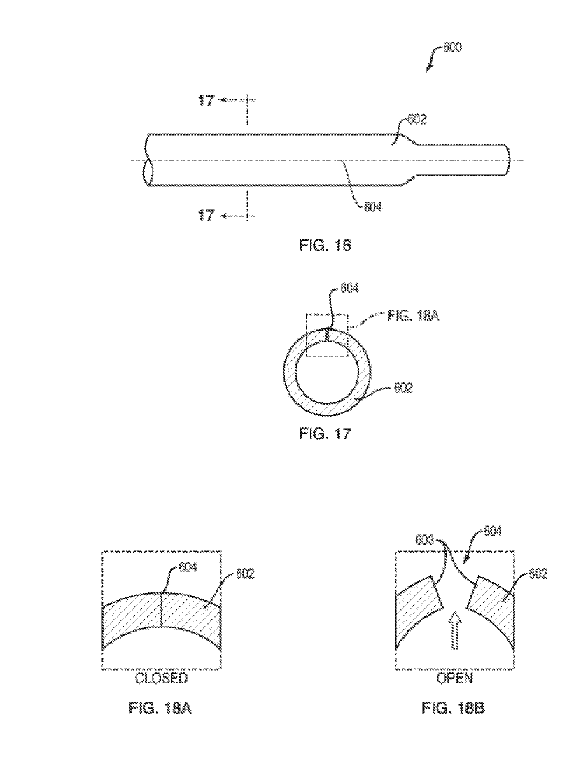

Referring to FIG. 16, a distal portion of a thrombolytic catheter 600 Is shown. The thrombolytic catheter 600 has a series of slits 604 that open once a threshold pressure level is reached within the lumen of the catheter 600. The slits may be disposed on the catheter shaft 602 in a number of varying configurations, as is known in the art. The slits 604 are integral to the catheter wall, and can be cut from a solid catheter shaft. The catheter shaft 602 may be formed from a compound polymer combining a relatively non-compliant medical grade polymer with a surface modifying fluoropolymer additive homogenously distributed throughout the wall of the catheter shaft 602 as illustrated in FIG. 17. This surface modifying fluoropolymer additive provides anti-thrombogenic and anti-adherent properties, as the surface modifiers tend to present on shear surfaces during extrusion of the catheter shaft. FIGS. 18A and 18B show a cross section of a slit in the closed and open configuration. Once silts 604 are cut into the catheter shaft, the fluoropolymer additive is automatically present and evenly distributed on the interior walls 603 of the slit. The additional and difficult step of uniformly coating the interior walls 603 of the slit is avoided, and a superior anti-fouling medical device is provided.

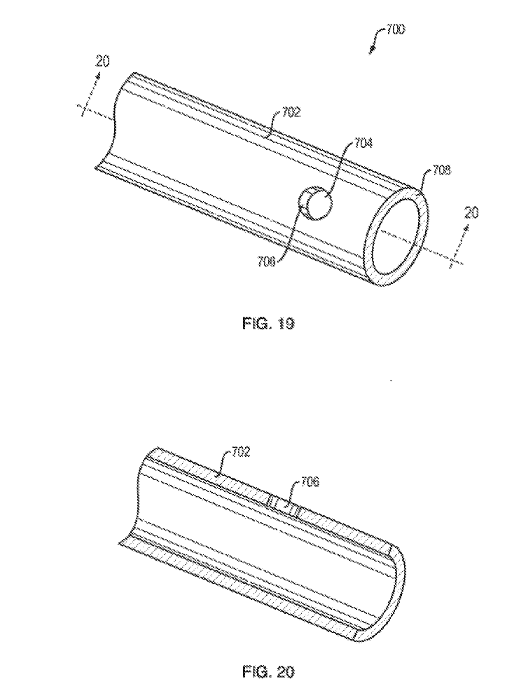

This simplified manufacturing technique for providing high performance thromboresistant and anti-adherent medical devices can be applied to numerous medical devices having side holes, for example, thrombolytic catheters, dialysis or drainage catheters. Referring to FIG. 19, a catheter 700 having a side hole 704 is shown. The catheter shaft 702 may be formed from an admixture of a base and a fluoropolymer additive homogenously distributed throughout the wall of the catheter shaft 702. The non-thrombogenic and anti-adherent surface modifying properties of the fluoropolymer additive present on shear surfaces during extrusion of the catheter shaft 702, and are also present at the surface of the catheter tip 708. After the catheter shaft 702 is formed, a side hole is cut into the shaft, and anti-adherent properties are instantly present on the entire inferior surface 706 of the side hole since the fluoropolymer additive is integral to the catheter shaft 702. As a result, the catheter can perform with a reduced incidence of side hole obstruction since platelets and thrombus are less likely to stick to and accumulate at the side holes. In addition, the distal end surface 708 will be less likely to exhibit fibrin sheath or thrombus build-up complications.

Urinary and drainage catheters of this design will have increased resistance to catheter encrustation or buildup of biofilms, often encountered during long-term urethral catheterization. The integral distribution of the fluopolymer additive in the shaft material provides for a finished product with anti-adherent properties on all surfaces, including the interior walls of the side and end holes, where encrustation and crystallization may tend to form and accumulate on the catheter. The minimization of these occurrences will lead to better device performance, lower infection rates, and lower health care costs as catheter replacement and remediation procedures are minimized. Further, these hard crystalline deposits can traumatize the patent upon catheter removal, and this design will help to minimize that risk of injury to the patient.

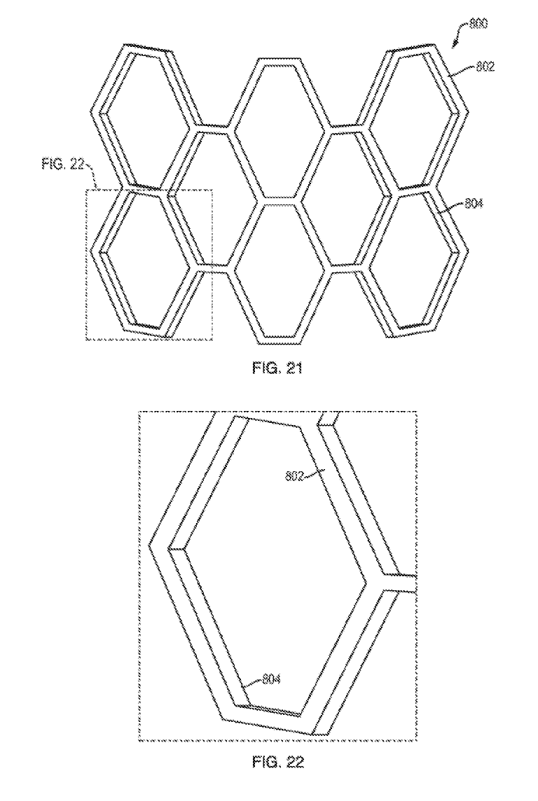

Referring now to FIGS. 21 and 22, a cross-pattern for an indwelling vascular device such as a stent of a filter is shown. The device can be made from an admixture polymer having a base polymer and a surface modifying fluoropolymer additive. Cross-pattern 800 has a combination of outward facing surfaces 802 and interior facing surfaces 804. If, for example, a similar cross pattern was adopted for use in a fluoropolymer coated stent, conventional methods of manufacturing the stent may involve forming the stent using a polymer or medical grade metal, and taking the additional step of coating the stent outward facing surfaces along with the inward facing surfaces. However, since the cross pattern illustrated according to the current embodiment is formed from a base polymer and fluoropolymer admixture having surface modifying properties, outward 802 and inward 804 facing surfaces are instantly thromboreslstant and anti-adherent since the fluoropolymer additive is integral throughout the bulk of each cross-pattern 800 member. Thus the adhesion of platelets and the accumulation of thrombus on cross-pattern 800 surfaces are minimized, minimizing risk of injury to the patient. Further, device performance is optimized, as inner surface areas and corners often most prone to platelet adhesion are anti-adherent. In addition, devices such as filters are often designed with members that press against or cut into the vessel wall for mounting and centering the device within the vessel lumen. Using the admixture in sections of the device that traumatize the vessel wall will facilitate non-traumatic removal of the device from the vessel lumen, where endothelial overgrowth and eventually restenosis of members contacting the vessel wall normally occurs. Further, manufacturing these types of devices is simplified because once the pattern is laser cut, the thromboresistant property is present in all outer and interior cut surfaces, saving the difficult step of evenly coating these surfaces. Further, durability is optimized since the properties will not elude over time.

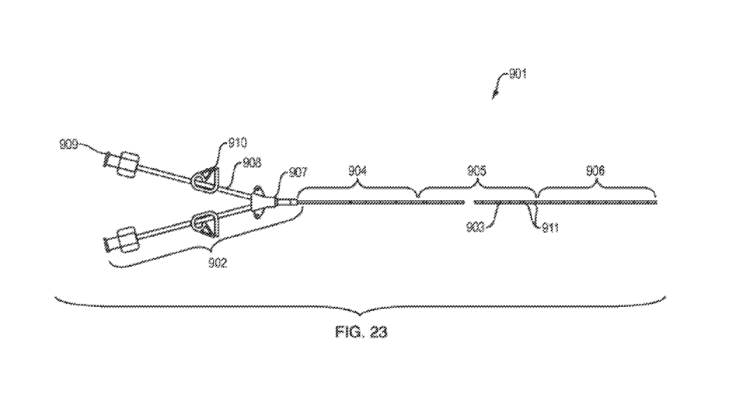

Referring to FIG. 23, a catheter (specifically a PICC line) according to an embodiment of the invention is shown. The catheter 901 is comprised of a hub section 902, a tube or shaft 903 with a proximal segment 904, a transition segment 905 and a distal segment 906. In the embodiment shown, a dual-lumen catheter is provided. A bifurcated hub component 907 and two extension legs 908 correspond to each shaft lumen. The extension legs 908 terminate at the proximal end with a connector such as a standard luer fitting 909 for connection to injection or aspiration devices. Leg clamps 910 coaxlally arranged around the extension legs 908 may be used to clamp off or occlude the leg lumens, preventing the inflow or outflow of fluids through the catheter 901. Alternatively, a proximal valve may he provided to prevent inflow and outflow of fluids through the device. The catheter may Include measurement markers 911 to assist in placement within the vessel. Although FIG. 23 depicts a PICC line, the present invention applies to other venous access devices including dialysis catheters, CVCs and implantable ports.

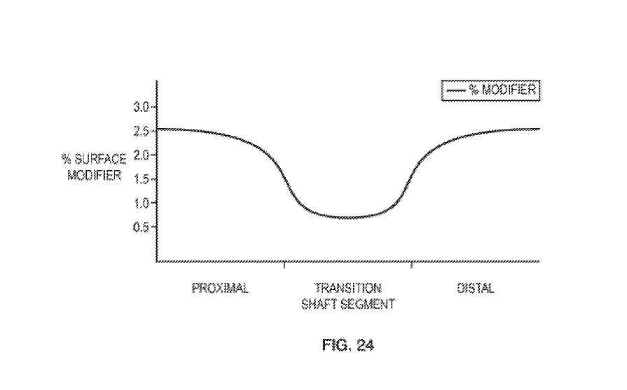

FIG. 24 illustrates a graph depicting the percentage by weight of the surface modifier additive relative to the polymer material along the length of a PICC catheter shaft. As shown in the graph and also referring to FIG. 23, at the proximal segment 904 of the tubing, the surface modifier additive comprises approximately 2.5% by weight of the shaft material. The percentage surface modifier additive decreases along the transitional segment to approximately 0.75% surface modifier by weight before increasing distally along the shaft to approximately 2.0%. The surface modifier additive ratio continues to increase along the distal segment rising from approximately 2.0% to 2.5% near the catheter tip section. Although the surface modifier/polymer base material mixture ranges from 0.75% to 2.5% in the graph shown, it is within the scope of the current invention to have a continuously varying ratio of modifier to polymer material of between 0.25% to 4.0%.

Although not illustrated, the surface modifier contained in each segment may transition more or less abruptly between segments. As an example, the entire proximal segment may contain an additive of 2.5% by weight which abruptly changes to 1.5% for the transition segment and 3.5% for the distal segment. Each segment may be separately extruded and then assembled together using RF welding or other known techniques to produce a monolithic catheter body. Alternatively, TIE extrusion as discussed below may be used to form the unitary shaft body.

A method of manufacturing a catheter shaft with varying ratios of materials along the length of the catheter shaft been previously described in U.S. Pat. No. 4,883,148 to Dandeneau, incorporated herein by reference. Specifically, the shaft tubing may be extruded using a Total Intermittent Extruded (TIE) process in which a polymer base is mixed with varying amounts of a second, different durometer material before being extruded as a single, unitary tube. The catheter shaft of the current embodiment may be manufactured using the TIE process to achieve the varying ratio of surface modifier to base polymer along the length of the catheter shaft. A method of manufacturing a venous access catheter shaft having customized and variable material characteristics of durometer and radiopaque filler has been previously described in U.S. Pat. No. 7,618,411 to Appling, Incorporated herein by reference.

Using the techniques described above, a venous access catheter shaft can be manufactured with sufficient surface modifier additive to prevent thrombus accumulation where it is most likely to occur thus maintaining the indwelling performance of the device. As a non-limiting example, the catheter shaft 903 may have higher concentrations of the surface modifier at the distal segment 906 of the shaft with a constant or varying percentage of surface modifier across the transition 905 and proximal 904 segments of the shaft. This design provides a catheter shaft having enhanced blood compatibility to prevent thrombus formation on the catheter tip section and also minimize the formation of a fibrin sheath tail at the distal tip of the catheter. As another non-limiting example, a venous access catheter shaft may be manufactured having sufficient surface modifier additive to prevent thrombus accumulation adjacent to the veno-puncture insertion site of the catheter. With reference back to FIG. 23, the catheter shaft 903 may have higher concentrations of the surface modifier at the proximal segment 904 of the shaft with a constant percentage of surface modifier across the transition 905 and distal segments 904. This design provides a catheter shaft having enhanced blood compatibility to prevent thrombus formation on the proximal 904 segment of the catheter shaft. This design may also prevent the formation and growth of a fibrin sheath along the outer surfaces of the catheter shaft surrounding the venous puncture site. Specifically, the surface modifier having fluorinated polymer composition results in a reduced friction catheter shaft surface. The reduced fouling character of the shaft surface and bulk prevents or significantly reduces the ability of thrombus or fibrin sheath to initially form on the catheter shaft. Because the thrombus and/or sheath does not form, there is no localized zone or nexus over which blofilm to adhere and infection to occur.

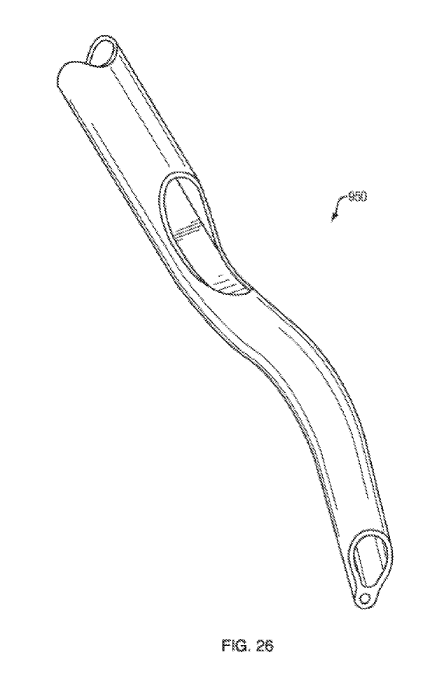

Providing a higher concentration of surface modifier along the distal 908 segment of the catheter shaft provides additional advantages when the distal tip section geometry of the shaft is non-uniform, irregular or non-tubular. For example, a dialysis catheter distal section is often shaped to maximize centering within the vessel and reduce side and end hole occlusions caused by aspiration during dialysis. See, for example FIG. 26 or U.S. Pat. No. 8,317,773 to Appling et al. incorporated by reference herein. The non-linear geometry of the catheter tip 950 shown in FIG. 26 creates flow turbulence and disruption within the vessel which in turn creates a nidus for blood adhesion when compared to tubes running straight along a common axis. In one aspect of the invention, blood adhesion at the distal section of the catheter may be reduced or eliminated by concentrating the surface modifier along non-linear tip sections of the dialysis catheter where thrombus is more likely to adhere. The percentage by weight of the surface modifier additive may be increased relative to the transition or middle section of the shaft.

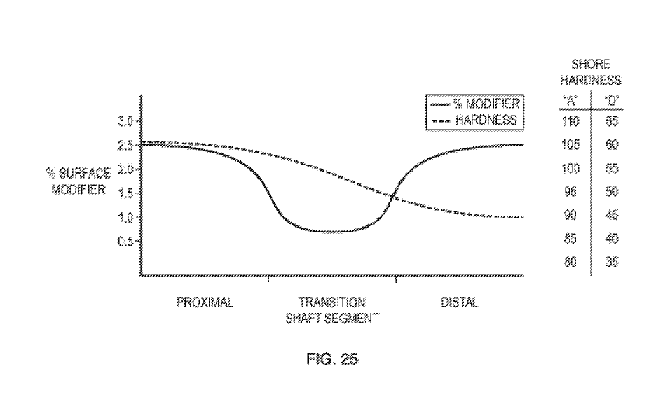

FIG. 25 illustrates another aspect of the invention which incorporates not only a varying amount of surface modifiers, but also polymer durometer hardness. Both the amount of surface modifier and durometer of the polymer material may be varied along the length of the shaft to produce a catheter having both enhanced anti-thrombogenic properties and shaft flexibility (material softness) at the distal segment. As can be seen in FIG. 25, at the proximal segment of the tubing, the mixture ratio is approximately 2.5% surface modifier (solid line) by weight with a relatively hard durometer (approximately 100 Shore A hardness) polymer material. As the shaft transitions from the proximal to transition segment of the shaft, both the durometer and surface modifier percentage level decrease. Moving from the transition shaft segment to the distal segment the durometer hardness continues to decrease to 85 A as the surface modifier begins to increase from approximately 2.0 to 2.5% along the length of the distal shaft segment.

Although not illustrated in FIG. 25, the amount of radiopaque filler may also be varied to produce a catheter shaft having customized characteristics including stiffness and tensile strength qualities. Stiffness and tensile strength are a function of the amount of radiopaque filler as well as the selected durometer of the polymer resin. In general the more radiopaque filler is present the lower the overall tensile strength of the shaft and the lower the durometer, the more flexible the shaft. As an example, the distal segment may include a higher concentration of both radiopaque filler and surface modifier to produce a shaft having increased visibility under imaging and enhanced resistance to blood platelet adhesion. Shaft flexibility may be enhanced by using a lower durometer polymer within the distal segment.

Accordingly, the radiopaque filler, the surface modifier and the base polymer percentages by weight may be varied to produce customized shaft qualities. As an example, the radiopaque filler may be increased along the transition segment until it reaches approximately 40% filler at the distal segment, the surface modifier may reach 2.5%, and the base polymer 57.5% by weight at the distal end section. The proximal segment may have a decreased amount of radiopaque filler and an increased amount of surface modifiers to produce a proximal shaft segment having both enhanced anti-thrombogenic characteristics and increased stiffness and strength due to the reduced percentage of radiopaque materials relative to polymer base. In general, by varying the urethane durometer, the amount by weight of radiopaque filler and the amount by weight of surface modifier additive, the desired flexural, tensile, radiopacity, thrombo-resistance and fibrin sheath resistance characteristics of the shaft may be customized to meet the specific clinical requirements.

Varying the concentration of the surface modifier along the catheter shaft also provides benefits associated with use of less surface modifier. The cost of a surface modifier additive is expensive and one way to minimize the costs associated with manufacture of the shaft is to use less overall surface modifier additive per a given length of tubing. As an example, once such benefit is lowered manufacturing costs due to using less overall surface modifier additive per catheter shaft by selectively concentrating the surface modifier where most needed and minimizing it where not needed. In addition, by minimizing the amount of surface modifier in those sections of the catheter shaft where it is least needed, the overall structural integrity and tensile strength of the polymer tube is not compromised.

In an alternative method of manufacturing the catheter shaft of the present invention, the monolithic tube is formed by joining two or more distinct tubing segments using an RF welding or thermal bonding procedure. This method is particularly advantageous for dialysis catheters with non-uniformly shaped tips, such as those disclosed in U.S. Pat. No. 8,317,773 to Appling et al. The transition and proximal tube segments may be formed from a base polymer and uniform percentage by weight of the surface modifier using a standard extrusion process. The percentage by weight of the surface modifier may range from 0% to 2.5% along the combined proximal/transition tube. The distal segment may be extruded separately and may be formed of a base polymer and surface modifier of a different percentage by weight from the other tubing segment. Typically, the surface modifier loading will be greater in the distal tip segment to provide enhanced platelet resistance at the catheter segment most likely to promote clot formation, as previously discussed. The monolithic tube is then formed by RF welding or thermal bonding the proximal end of the distal segment to the distal end of the proximal segment to create a seamless, unitary shaft. The RF welding or thermal bonding processes may also include forming the tip geometry. The resultant monolithic tube is characterized by a proximal and transition segment having a uniform surface modifier loading across the combined length of the two segments and a distal segment with a greater surface modifier loading with a non-linear tip profile. The distal segment surface loading may be uniform across the segment length or may be varied as previously described.

1-11. (canceled)

12. A vascular access device, comprising:
a port assembly;
a reservoir arranged within the port assembly and configured to receive a fluid from a vascular system, at least a portion of the reservoir lined with an insert.

13. The vascular access device of claim 12, the insert comprising an admixture of a base polymer and a surface modifying fluoropolymer additive.

14. The vascular access device of claim 12, the reservoir fluidly sealed by a septum.

15. The vascular access device of claim 12, the at least a portion comprising a bottom portion and a side portion of the reservoir.

16. The vascular access device of claim 12, the at least a portion comprising a portion of the reservoir bounded by a base housing portion of the port assembly.

17. The vascular access device of claim 12, the reservoir comprising a top portion sealed by a septum and a remaining portion lined by the insert.

18. The vascular access device of claim 12, the insert formed as a floor insert arranged to cover a bottom surface of the reservoir.

19. The vascular access device of claim 18, at least a portion of side-walls of the reservoir not covered by the floor insert comprising an admixture of a base polymer and a surface modifying fluoropolymer additive.

20. The vascular access device of claim 18, the floor insert having a soft durometer to stabilize a needle inserted into the floor insert.

21. The vascular access device of claim 12, the insert comprising an opening arranged to provide fluid communication between the reservoir and an outlet lumen.

22. The vascular access device of claim 12, the insert comprising an integral outlet lumen.

23. The vascular access device of claim 13, the base polymer comprising silicone.

24. The vascular access device of claim 13, the surface modifying fluoropolymer additive comprising a 2-25% by weight ratio of additive to base polymer.

25. The vascular access device of claim 13, the surface modifying fluoropolymer additive is homogenously distributed throughout a surface of the admixture.

26. A vascular access device, comprising:
a port assembly;
a reservoir arranged within the port assembly and configured to receive a fluid from a vascular system, at least a portion of the reservoir formed of a surface-modifying macromolecule.

27. The vascular access device of claim 26, the surface-modifying macromolecule comprising:
i) a non-chain extended central portion of a segmented block copolymer having a molecular weight of less than 2,000 and comprising a total of no more than ten units A and B, wherein A is a urethane containing group; and B is selected from the group consisting of a polyalkylene oxide, polycarbonate, polyester and lactone; and
ii) α-ω terminal polyfluoro oligomeric groups.

28. The vascular access device of claim 26, the surface-modifying macromolecule comprising:
iii) a non-chain extended central portion of a segmented block copolymer having a molecular weight of less than 5,000 and comprising a total of no more than ten monomelic units A and B, wherein A is a urethane containing group; and B is selected from the group consisting of a polyalkylene oxide, polycarbonate, polyester and lactone; and
iv) α-ω terminal polyfluoro oligomeric groups, selected from CF₃(CF₂)ₚCH₂CH₂, wherein p is 5-9 and CF₃(CF₂)ₘ(CH₂CH₂O)ₙ, wherein n is 1-10 and m is 1-20.

29. A method of manufacturing a vascular access device, comprising:
providing a port assembly;
arranging a reservoir within the port assembly configured to receive a fluid from a vascular system;
lining at least a portion of the reservoir with an insert.

30. The method of claim 29, the insert comprising an admixture of a base polymer and a surface modifying fluoropolymer additive.

31. The method of claim 29, the at least a portion comprising a portion of the reservoir bounded by a base housing portion of the port assembly.
